(19) United States
(12) Patent Application Publication
(10) Pub. No.: US 2025/0339080 A1
(43) Pub. Date: Nov. 6, 2025

(54) SYSTEM AND METHOD FOR ELECTROPHYSIOLOGICAL MAPPING

(71) Applicant: St. Jude Medical, Cardiology Division, Inc., St. Paul, MN (US)

(72) Inventors: Don Curtis Deno, Andover, MN (US); Sofia Fardella, Minneapolis, MN (US)

(21) Appl. No.: 19/194,781

(22) Filed: Apr. 30, 2025

Related U.S. Application Data
(60) Provisional application No. 63/643,088, filed on May 6, 2024.

(51) Int. Cl. A61B 5/367; A61B 5/287; A61B 5/341

(52) U.S. Cl. CPC A61B 5/367; A61B 5/287; A61B 5/341

(57) ABSTRACT

Electrophysiological activity can be mapped using an electroanatomical mapping system. Using electrophysiological data from a clique of at least four non-coplanar electrodes, the mapping system derives a three-dimensional vectorcardiogram for the clique; analyzes a shape of the vectorcardiogram; identifies first and second omnipolar electrograms for the clique; defines an activation direction for the clique; and computes a conduction velocity magnitude for the clique, thereby determining a cardiac activation vector at the cardiac location. The cardiac location can be classified as pathological when the orientations of the first and second omnipolar electrograms differ by more than a threshold amount and/or when the shape of the three-dimensional vectorcardiogram satisfies at least one of a non-planarity criterion and a directional criterion. Various graphical representations of the foregoing analyses are contemplated.

### CROSS-REFERENCE TO RELATED APPLICATIONS

The instant application claims the benefit of U.S. provisional application No. 63/643,088, filed 6 May 2024, which is hereby incorporated by reference as though fully set forth herein.

### FIELD

The present disclosure relates generally to electrophysiological mapping, as may be performed in cardiac diagnostic and therapeutic procedures. In particular, the present disclosure relates to systems, apparatuses, and methods for measuring cardiac activation directions in three-dimensions. That is, the present disclosure does not constrain measurements of cardiac activation to along the cardiac surface, but also contemplates measuring transmural components of cardiac activation.

### BACKGROUND

Electrophysiological mapping, and more particularly electrocardiogramapping, is a part of numerous cardiac diagnostic and therapeutic procedures. Such mapping includes measuring the electrical activity of the heart over time using surface leads and/or intracardiac measurement electrodes. The voltage-over-time waveforms measured by surface leads are commonly referred to as electrocardiograms (ECG or EKG), while those measured by intracardiac measurement electrodes are commonly referred to as electrograms (EGM).

Electrophysiology studies often include mapping the activation wavefront as it propagates along the cardiac surface, because visualizations of activation maps can provide insight to a practitioner as to how an arrhythmia is traveling through the cardiac chambers and where ablation therapy may be applied in order to terminate the arrhythmia.

Extant approaches to mapping cardiac activation wavefronts, however, tend to be constrained to two-dimensional analysis. That is, the cardiac activation vectors that are computed are limited to propagation along the cardiac surface, with the assumption that the same propagation is observed at any point through the myocardium.

In reality, however, cardiac activation is not uniform through the myocardium. Instead, cardiac activation can also proceed transmurally-through the cardiac wall instead of, or in addition to, along the cardiac surface.

### BRIEF SUMMARY

The instant disclosure provides a method of mapping cardiac electrophysiological activity including: receiving, at an electroanatomical mapping system, electrophysiological data from at least four non-coplanar electrodes positioned at a cardiac location, the at least four non-coplanar electrodes defining a three-dimensional electrode clique; and the electroanatomical mapping system executing a process that includes the steps of: deriving a three-dimensional vectorcardiogram for the three-dimensional electrode clique; analyzing a shape of the three-dimensional vectorcardiogram; identifying a first omnipolar electrogram for the three-dimensional electrode clique, wherein the first omnipolar electrogram is a maximum peak-to-peak voltage omnipolar electrogram; identifying a second an omnipolar electrogram for the three-dimensional electrode clique, wherein the second omnipolar electrogram has a best morphological match to a unipolar electrogram for the three-dimensional electrode clique; using at least one of an orientation of the first omnipolar electrogram and an orientation of the second omnipolar electrogram to define an activation direction for the three-dimensional electrode clique; and computing a conduction velocity magnitude for the three-dimensional electrode clique, thereby determining a cardiac activation vector at the cardiac location.

The process executed by the electroanatomical mapping system can also include classifying the cardiac location as pathological when the orientation of the first omnipolar electrogram differs from the orientation of the second omnipolar electrogram by more than a threshold amount.

The process executed by the electroanatomical mapping system can also include classifying the cardiac location as pathological when the shape of the three-dimensional vectorcardiogram satisfies at least one of a non-planarity criterion and a directional criterion. For instance, analyzing the shape of the three-dimensional vectorcardiogram can include analyzing at least one of planarity of the three-dimensional vectorcardiogram and an angle of the three-dimensional vectorcardiogram relative to a cardiac surface at the cardiac location. In turn, the planarity of the three-dimensional vectorcardiogram can be analyzed using singular value decomposition.

It is contemplated that the method can include the electroanatomical mapping system outputting a graphical representation of the cardiac activation vector at the cardiac location. For instance, the electroanatomical mapping system can output the graphical representation of the cardiac activation vector at the cardiac location when the cardiac activation vector satisfies a transmural conduction criterion, such as a threshold for an angle between the activation direction and a cardiac surface at the cardiac location.

The at least four non-coplanar electrodes can include a segmented tip electrode and a ring electrode carried by a multi-electrode catheter, wherein the segmented tip electrode includes at least three segments. Alternatively, the at least four non-coplanar electrodes can include a tip electrode and a segmented ring electrode carried by a multi-electrode catheter, wherein the segmented ring electrode includes at least three segments. The at least four non-coplanar electrodes can include a segmented tip electrode and a segmented ring electrode carried by a multi-electrode catheter.

Also disclosed herein is an electroanatomical mapping system for generating a cardiac activation map. The electroanatomical mapping system includes a cardiac activation module configured to: receive electrophysiological data from at least four non-coplanar electrodes positioned at a cardiac location, the at least four non-coplanar electrodes defining a three-dimensional electrode clique; derive a three-dimensional vectorcardiogram for the three-dimensional electrode clique; analyze a shape of the three-dimensional vectorcardiogram; identify a first omnipolar electrogram for the three-dimensional electrode clique, wherein the first omnipolar electrogram is a maximum peak-to-peak voltage omnipolar electrogram;

identify a second omnipolar electrogram for the three-dimensional electrode clique, wherein the second omnipolar electrogram has a best morphological match to a unipolar electrogram for the three-dimensional electrode clique; define an activation direction for the three-dimensional electrode clique using at least one of an orientation of the first omnipolar electrogram and an orientation of the second omnipolar electrogram; compute a conduction velocity magnitude for the three-dimensional electrode clique; and associate the activation direction and the conduction velocity magnitude as a cardiac activation vector at the cardiac location.

In aspects of the disclosure, the cardiac activation module is further configured to classify the cardiac location as pathological when the orientation of the first omnipolar electrogram differs from the orientation of the second omnipolar electrogram by more than a threshold amount.

In further aspects of the disclosure, the cardiac activation module is further configured to classify the cardiac location as pathological when the shape of the three-dimensional vectorcardiogram satisfies at least one of a non-planarity criterion and a directional criterion. For instance, the cardiac activation module can be configured to analyze the shape of the three-dimensional vectorcardiogram by analyzing at least one of a planarity of the three-dimensional vectorcardiogram (e.g., using singular value decomposition) and an angle of the three-dimensional vectorcardiogram relative to a cardiac surface at the cardiac location.

It is also contemplated that the cardiac activation module can be configured to output a graphical representation of the cardiac activation vector at the cardiac location. The graphical representation of the cardiac activation vector at the cardiac location can include an arrow icon superimposed upon a three-dimensional model of a cardiac geometry and may be output only when the cardiac activation vector satisfies a transmural conduction criterion, such as a threshold for an angle between the activation direction and a cardiac surface at the cardiac location.

There is also provided a computer-readable medium, a record carrier, or a computer program product comprising instructions that, when executed, cause a computer or processor to perform any of the methods set forth herein. It will also be appreciated that the methods undertaken herein, including the various derivations and computations may be undertaken by a processor or a computer on data representative of the received signals, for example, the received voltages sensed by each of the plurality of electrodes.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIGS. 2A-2D depict various exemplary multi-electrode catheter embodiments that can be used in connection with aspects of the instant disclosure.

FIG. 3 is a flowchart of representative steps that can be carried out according to aspects of the instant disclosure.

FIG. 4A depicts representative vectorcardiogram loops for a clique of four or more non-coplanar electrodes from a pair of successive depolarizations.

FIGS. 4B-4D depict vectorcardiogram time interval plots for cliques of four or more non-coplanar electrodes from single depolarizations.

FIG. 5 is a representative contour plot of angle-dependent morphological correspondence between omnipolar electrograms for a clique of four or more non-coplanar electrodes and a unipolar time derivative electrogram for that same clique.

FIG. 6 is a representative contour plot of angle-dependent peak-to-peak voltage of omnipolar electrograms for a clique of four or more non-coplanar electrodes.

FIGS. 7A through 7C are representative graphical outputs of cardiac activation vectors (arrow icons and similar glyphs) superimposed upon a three-dimensional model of the cardiac geometry from anterior and superior-lateral views.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for mapping electrophysiological activity in three dimensions. For purposes of illustration, aspects of the disclosure will be described with reference to cardiac electrophysiological mapping using a linear multi-electrode catheter in conjunction with an electroanatomical mapping system, such as the EnSite Precision™ cardiac mapping system (Abbott Laboratories, Abbott Park, IL). Exemplary linear multi-electrode catheters include, without limitation, the TactiFlex™ Ablation Catheter, Sensor Enabled™ (Abbott Laboratories); the TactiCath™ Contact Force Ablation Catheter, Sensor Enabled™ (Abbott Laboratories); the Safire™ Ablation Catheter (Abbott Laboratories); QDOT Micro™ radiofrequency ablation catheter (Biosense Webster, Inc., Diamond Bar, CA); and the Intellatip MIFI™ XP temperature ablation catheter (Boston Scientific Corporation, Marlborough, MA). Those of ordinary skill in the art will understand, however, how to apply the teachings herein to good advantage in other contexts and/or with respect to other devices.

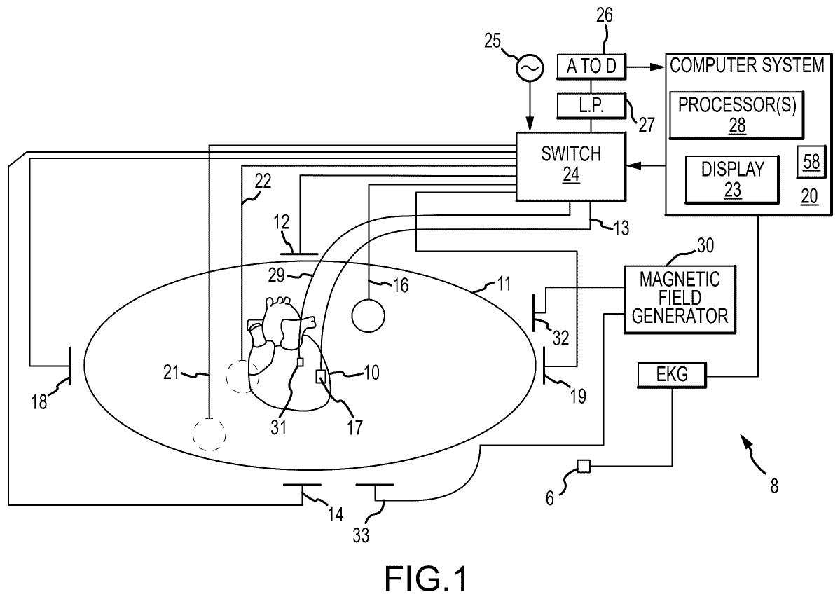

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10.

As one of ordinary skill in the art will recognize, system 8 determines the location, and in some aspects the orientation, of objects, typically within a three-dimensional space, and expresses those locations as position information determined relative to at least one reference. This is referred to herein as “localization.”

For simplicity of illustration, the patient 11 is depicted schematically as an oval. In the embodiment shown in FIG. 1, three sets of surface electrodes (e.g., patch electrodes) 12, 14, 16, 18, 19, and 22 are shown applied to a surface of the patient 11, pairwise defining three generally orthogonal axes, referred to herein as an x-axis (12, 14), a y-axis (18, 19), and a z-axis (16, 22). In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 16, 22 are applied to the patient along a second axis generally orthogonal to the x-axis, along the sternum and spine of the patient in the thorax region, and may be referred to as the Chest and Back electrodes. The z-axis electrodes 18, 19 are applied along a third axis generally orthogonal to both the x-axis and the y-axis, such as along the inner thigh and neck regions of the patient, and may be referred to as the Left Leg and Neck electrodes. The heart 10 lies between these pairs of surface electrodes 12/14, 16/22, and 18/19.

Each surface electrode can measure multiple signals. For example, in embodiments of the disclosure, each surface electrode can measure complex impedance as three resistance signals and three reactance signals. These signals can, in turn, be grouped into three resistance/reactance signal pairs. One resistance/reactance signal pair can reflect driven values, while the other two resistance/reactance signal pairs can reflect non-drive values (e.g., measurements of the electric field generated by other driven pairs in a manner similar to that described below for electrodes 17).

An additional surface reference electrode (e.g., a “belly patch”) 21 provides a reference and/or ground electrode for the system 8. The belly patch electrode 21 may be an alternative to a fixed intra-cardiac electrode 31, described in further detail below. In alternative embodiments where system 8 is capable of magnetic field-based localization instead of or in addition to impedance-based localization, the surface electrode 21 can alternatively or additionally include a magnetic patient reference sensor-anterior (“PRS-A”) positioned on the patient's chest.

It should be appreciated that patient 11 may also have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. In other embodiments, system 8 may utilize a single catheter that includes multiple (e.g., eight) splines, each of which in turn includes multiple (e.g., eight) electrodes.

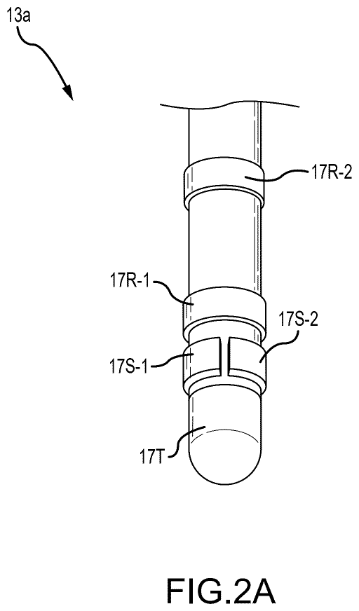

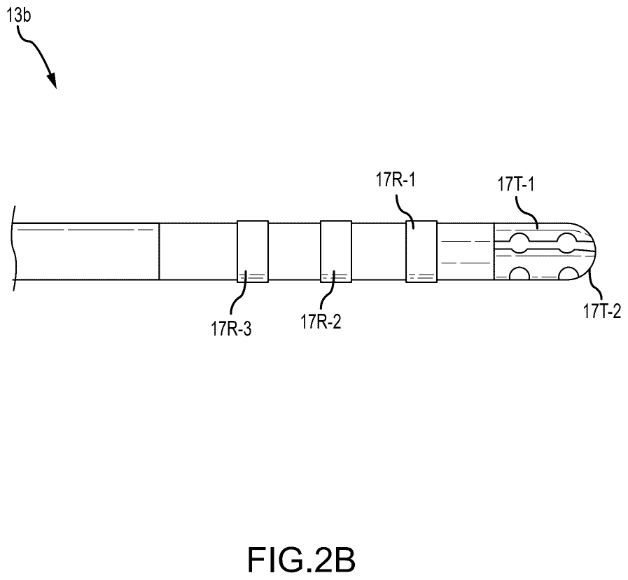

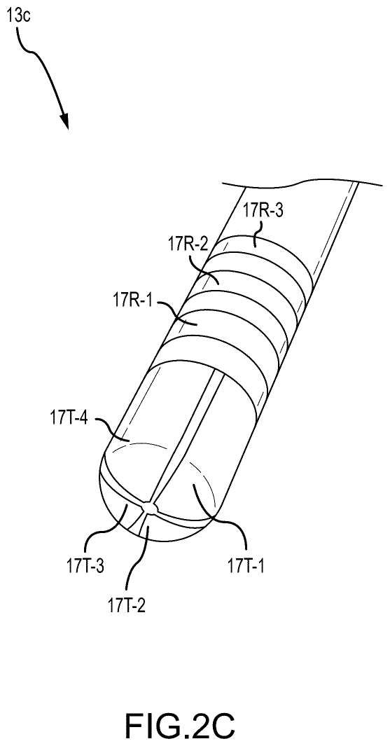

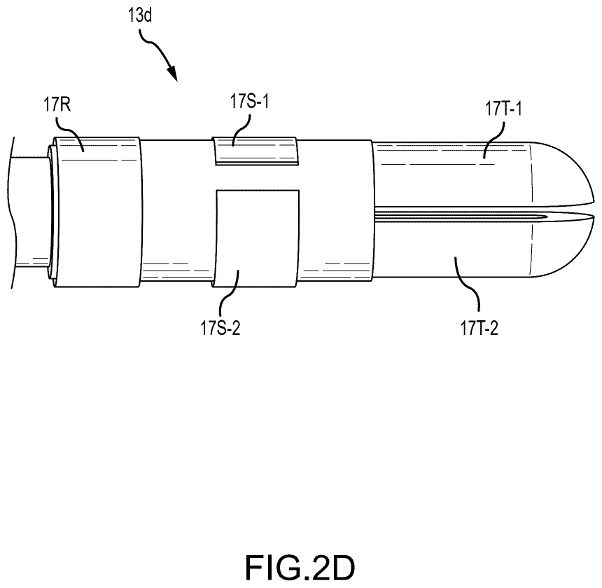

The foregoing embodiments are merely exemplary, however, and any number of electrodes and/or catheters may be used. For example, for purposes of this disclosure, the distal portions of various exemplary multi-electrode catheters are shown in FIGS. 2A-2D.

FIG. 2A depicts the distal portion of a first exemplary catheter 13a including a plurality of electrodes disposed within its distal portion. More particularly, catheter 13a includes a tip electrode 17T and a segmented ring electrode having three segments, each of which extends about one-third of the way around the perimeter of catheter 13a. Segments 17S-1 and 17S-2 are visible, while the third segment (referred to as 17S-3) is hidden on the reverse side of the view of FIG. 2A. Catheter 13a also carries two additional ring electrodes, 17R-1 and 17R-2.

FIG. 2B depicts the distal portion of a second exemplary catheter 13b including a plurality of electrodes disposed within its distal portion. More particularly, catheter 13b includes a segmented tip electrode having three segments, each of which extends about one-third of the way around the perimeter of catheter 13b. Segments 17T-1 and 17T-2 are visible, while the third segment (referred to as 17T-3) is hidden on the reverse side of the view of FIG. 2B. Catheter 13b also carries a plurality of ring electrodes 17R-1, 17R-2, and 17R-3.

FIG. 2C depicts the distal portion of a third exemplary catheter 13c including a plurality of electrodes disposed within its distal portion. More particularly, catheter 13c includes a segmented tip electrode having four segments 17T-1 through 17T-4, each of which extends about one-quarter of the way around the perimeter of catheter 13c. Catheter 13c also carries a plurality of ring electrodes 17R-1, 17R-2, and 17R-3.

FIG. 2D depicts the distal portion of a fourth exemplary catheter 13d including a plurality of electrodes disposed within its distal portion. More particularly, catheter 13d includes a segmented tip electrode having two segments 17T-1 and 17T-2, each of which extends about halfway around the perimeter of catheter 13d. Catheter 13d also carries a segmented ring electrode having three segments, each of which extends about one-third of the way around the perimeter of catheter 13d. Segments 17S-1 and 17S-2 are visible, while the third segment (referred to as 17S-3) is hidden on the reverse side of the view of FIG. 2D. Catheter 13d also carries a ring electrode 17R.

It should be understood that the configurations shown in FIGS. 2A-2D are merely exemplary. The number, configuration, and placement of electrodes may vary without departing from the scope of the instant disclosure. In connection with the present teachings, however, it is desirable for electrodes 17 to be substantially arranged in one dimension (e.g., arranged along the longitudinal axis of a linear catheter 13). Because such catheters are relatively thin, at least in their distal regions where electrodes 17 are located, they may be better able to access certain regions of the cardiac anatomy (e.g., pulmonary veins, the saddle shaped carina, and around papillary muscles) than, for example, grid catheters (e.g., catheters where the electrodes are arranged in a plane), basket catheters, and other two-and three-dimensional catheters.

Moreover, it is contemplated that, in some embodiments of the disclosure, the same catheter 13 is used for both diagnostic (e.g., electrophysiological mapping) and therapeutic (e.g., radiofrequency (RF) ablation and/or pulsed field ablation (PFA)) procedures. According to these embodiments of the disclosure, the segmented electrodes are sufficiently closely-spaced that, for purposes of ablation, they are effectively shorted together (e.g., segmented electrodes 17S-1, 17S-2, and 17S-3 of FIG. 2A operate as a single ring electrode analogous to ring electrode 17-R1). On the other hand, the segmented electrodes are sufficiently spaced apart that, for purposes of pacing or sensing electrophysiological signals (further discussed below), they are separate. Those of ordinary skill in the art will be familiar with appropriate inter-electrode spacings to achieve these objects.

These dual-use electrode configurations offer advantages. For example, they reduce or eliminate the need to exchange catheters during a procedure (e.g., mapping with a first catheter and then ablating with a second catheter), in turn reducing the time and complexity of an overall electrophysiology procedure. They also mitigate variations in localization that might occur as between different localization sensors on different catheters, even though the catheters may be in the same physical location within the subject's heart.

As the ordinarily-skilled artisan will appreciate, electrodes 17 carried in the distal portion of catheter 13 can be used to measure unipolar electrograms. Unipolar electrograms have the advantage of being orientation-independent (that is, a given electrode 17 will measure substantially the same unipolar electrogram regardless of the orientation of catheter 13 relative to the cardiac surface). On the other hand, unipolar electrograms often include not only the component of interest (e.g., a near-field cardiac activation component), but also various far-field noise components. These far-field noise components include, but are not limited to, far-field cardiac activation components, powerline noise components, patient respiration components, patient motion components, and cardiac motion components. Unipolar electrograms also ignore local directional information in the form of the ion currents that produce electrograms.

It may also be desirable to measure bipolar electrograms, which are less susceptible to far-field noise than unipolar electrograms and incorporate directional information along their axes. As those of ordinary skill in the art will recognize, any two neighboring electrodes 17 on catheter 13 define a bipole. Any bipole can, in turn, be used to generate a bipolar electrogram according to techniques that will be familiar to those of ordinary skill in the art. For instance, referring to FIG. 2A, the bipolar electrogram for the bipole defined by electrodes 17S-1 and 17S-2 can be computed as the difference of the 17S-1 and 17S-2 unipolar electrograms, with the common mode rejection that results from this operation mitigating the impact of far-field noise in the bipolar electrogram. Those of ordinary skill in the art will recognize, however, that bipolar electrograms are orientation-dependent (that is, they will change as the orientation of catheter 13 relative to the cardiac surface changes).

There are, however, extant techniques that allow bipolar electrograms to be combined to generate electrograms for any orientation of catheter 13 relative to the cardiac surface without physically changing the orientation of catheter 13. Such techniques are often referred to as “orientation-independent” or “omnipolar” techniques. In turn, the computed electrogram signals that result from the application of such techniques can be referred to as “omnipolar electrograms” or “virtual bipolar electrograms.” These omnipolar electrograms can be thought of as the bipolar electrogram that would be seen by an “omnipole” or “virtual bipole” having its “omnipole orientation” or “virtual bipole orientation” at a particular angle relative to the cardiac anatomy (e.g., the cardiac surface).

In embodiments of the disclosure, omnipolar techniques are applied to the several unipolar and bipolar electrograms that can be measured by a three-dimensional clique of four or more non-coplanar electrodes. Electrodes may be grouped together in clusters or “cliques” (of four or more electrodes) to allow for measurement of multiple signals. A “clique” may be a group of electrodes that are offset at the vertices of a tetrahedron (e.g., four electrodes in close proximity to one another on the distal portion of a catheter). For instance, referring to FIG. 2A, electrodes 17T, 17S-1, 17S-2, and 17S-3 form a first tetrahedral clique, while electrodes 17S-1, 17S-2, 17S-3, and 17R-1 form a second tetrahedral clique. The electrodes within each clique can be used to measure various unipolar and bipolar electrograms. For instance, each individual electrode can be used to measure a unipolar electrogram; those of ordinary skill in the art will be familiar with various techniques suitable for defining a representative unipolar electrogram for the clique as a whole (e.g., an average of the four individual unipolar electrograms). Likewise, each pair of electrodes within a clique can be used to measure a bipolar electrogram.

Cliques and clusters can be used to detect the voltage and real-time wavefront direction and speed independent of catheter orientation and can be used for “omnipolar” mapping. A clique or cluster of at least four electrodes positioned in close proximity allows for the measurement of multiple signals (rather than, for example, on a single axis when measurements are taken from electrodes placed along the length of the catheter).

Details of computing E-field loops from the various unipolar and bipolar electrograms that can be measured using three-dimensional electrode cliques, and for generating omnipolar electrograms therefrom, are described in U.S. Pat. Nos. 10,758,137 and 10,194,994; international patent application publication no. WO 2015/130824; and Deno et al., Orientation-Independent Catheter-Based Characterization of Myocardial Activation, IEEE Transactions on Biomedical Engineering, Vol. 64, No. 5, 1067-1077 (May 2017) (“Deno”). Each of the foregoing is hereby incorporated by reference as though fully set forth herein.

As will be apparent from the foregoing description, catheter 13 can be used to simultaneously collect a plurality of electrophysiology data points for the various unipoles and bipoles defined by electrodes 17 thereon. Each such electrophysiology data point includes both localization information (e.g., position of a unipole; position and orientation of a selected bipole or electrode clique) and corresponding electrogram signals (e.g., unipolar, bipolar, and/or omnipolar electrograms). For purposes of illustration, methods according to the instant disclosure will be described with reference to individual electrophysiology data points collected by catheter 13. It should be understood, however, that the teachings herein can be applied, in serial and/or in parallel, to multiple electrophysiology data points collected by catheter 13 (e.g., over a plurality of cliques for a given position of catheter 13 within the heart, as well as for various positions of catheter 13 within the heart).

Catheter 13 (or multiple such catheters) are typically introduced into the heart and/or vasculature of the patient via one or more introducers and using familiar procedures. Indeed, various approaches to introduce catheter 13 into a patient's heart, such as transseptal approaches, will be familiar to those of ordinary skill in the art, and therefore need not be further described herein.

Since each electrode 17 lies within the patient, location data may be collected simultaneously for each electrode 17 by system 8. Similarly, each electrode 17 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate graphical representations of cardiac geometry and/or cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein. Further, the methods and processes described herein may be embedded within a set of human-or machine-readable instructions that are comprised within a computer-readable medium or record carrier, or that are comprised within a computer program product. The instructions are such that, when executed by a computer or processor, the computer or processor causes the system to perform the methods described herein.

Generally, three nominally orthogonal electric fields are generated by a series of driven and sensed electric dipoles (e.g., surface electrode pairs 12/14, 16/22, and 18/19) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from navigational currents and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data from the surface electrodes and/or the internal electrodes may be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current signals. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 8 can be a hybrid system that incorporates both impedance-based (e.g., as described above) and magnetic-based localization capabilities. Thus, for example, system 8 can also include a source 30 coupled to one or more magnetic field generators. In the interest of clarity, only two magnetic field generators 32 and 33 are depicted in FIG. 1, but it should be understood that additional magnetic field generators (e.g., a total of six magnetic field generators, defining three generally orthogonal axes analogous to those defined by patch electrodes 12, 14, 16, 18, 19, and 22) can be used without departing from the scope of the present teachings. Likewise, those of ordinary skill in the art will appreciate that, for purposes of localizing catheter 13 within the magnetic fields so generated, can include one or more magnetic localization sensors (e.g., coils).

In some embodiments, system 8 is the EnSite™ X, EnSite™ Velocity™, or EnSite Precision™ electrophysiological mapping and visualization system of Abbott Laboratories. Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stercotaxis, Inc.'s NIOBE® Magnetic Navigation System (St. Louis, Missouri), the Affera™ Mapping and Ablation System of Medtronic plc (Minneapolis, Minnesota), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983, 126; and 5,697,377.

Aspects of the disclosure relate to generating electrophysiology maps and, in particular, cardiac activation maps that aid a practitioner in identifying focal sources, connections, and other arrhythmia-sustaining pathologies. Such regions are often desirable targets for ablation therapy. System 8 (e.g., computer system 20) can therefore include a hardware-and/or software-based cardiac activation module 58 as further described below.

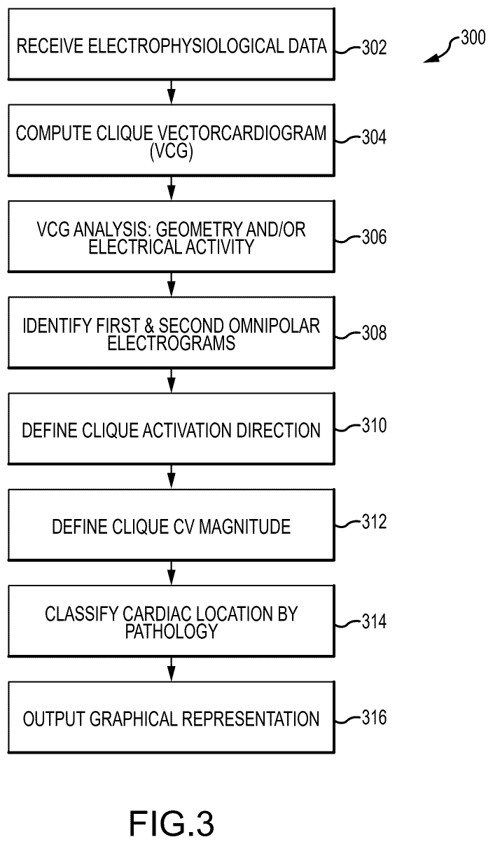

Exemplary methods according to aspects of the instant disclosure will be explained with reference to the flowchart 300 of representative steps presented as FIG. 3. In some embodiments, for example, flowchart 300 may represent several exemplary steps that can be carried out by electroanatomical mapping system 8 of FIG. 1 (e.g., by processor 28 and/or cardiac activation module 58). It should be understood that the representative steps described below can be hardware-implemented, software-implemented, or implemented in a combination of hardware and software.

In block 302, system 8 receives electrophysiological data from at least four non-coplanar electrodes 17 on multi-electrode catheter 13 that, as described above, define a three-dimensional electrode clique. For instance, system 8 can receive a unipolar electrogram signal from each electrode 17 within the clique; system 8 can also compute one or more bipolar electrograms (including omnipolar electrograms) therefrom.

As described above, system 8 can localize electrodes 17 and/or multi-electrode catheter 13 using techniques that will be familiar to those of ordinary skill in the art. Thus, the electrophysiological data (e.g., the received unipolar electrograms and/or the bipolar electrograms computed therefrom) can be associated with the cardiac location at which they were measured and/or computed. The association of electrophysiological data (e.g., electrogram signals) with the cardiac location at which such data were gathered (or computed) is commonly referred to as an “electrophysiology data point” (or “EP data point”). A collection of EP data points can be used to define an “electrophysiology map,” which, in turn, can be output to display 23 of system 8. It will also be appreciated that system 8, and more specifically, computer 20 is configured to perform determinations, derivations, and calculations on electrophysiology data points that are representative of various signals sensed at the electrodes.

In block 304, system 8 derives a three-dimensional vectorcardiogram for the electrode clique. Vectorcardiography is a method of recording the magnitude and direction of the electrical forces that are generated by the heart using a continuous series of vectors that form curving lines around a central point.

System 8 analyzes the clique vectorcardiogram in block 306. Analysis of the clique vectorcardiogram according to techniques disclosed herein can offer a practitioner insight into whether the tissue at the cardiac location at which the electrophysiological data were measured is pathological.

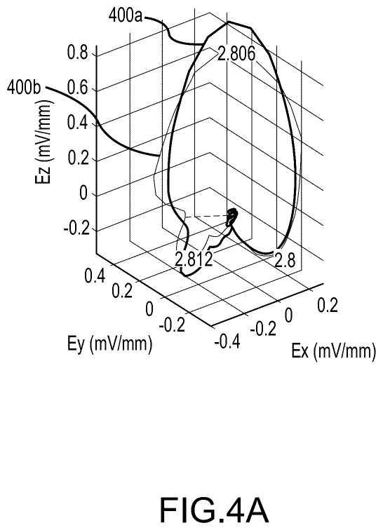

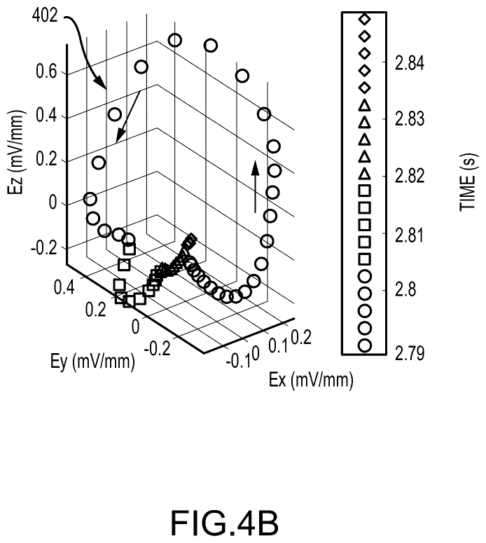

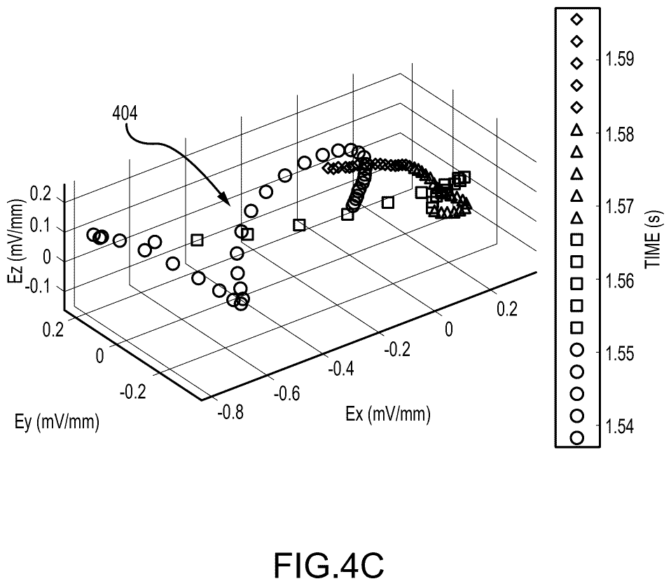

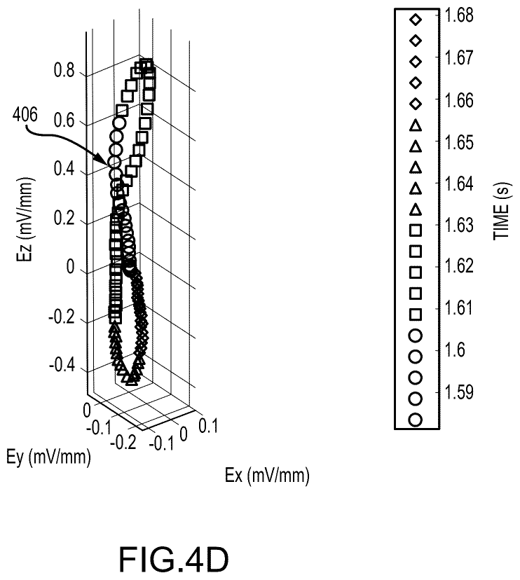

In a first aspect of block 306, system 8 analyzes the shape and/or geometry of the clique vectorcardiogram. This can be understood with reference to FIGS. 4A-4D. FIG. 4A illustrates vectorcardiogram loops 400a, 400b for two successive beats, with the time in seconds since the start of the respective loop annotated thereon (note that substantially all of the three-dimensional depolarization loop occurs in about a 12 millisecond interval from about 2.800seconds to about 2.812 seconds). FIG. 4B plots (402) vectorcardiogram loop 400b in 0.5 ms intervals. Similar time interval plots (404, 406) are shown in FIGS. 4C and 4D.

Two aspects of the shape and/or geometry of the clique vectorcardiogram (or corresponding time interval plot) of particular interest are the planarity of the vectorcardiogram and the direction(s) of the vectorcardiogram (that is, the angle(s) of the vectorcardiogram relative to the cardiac surface). A highly planar vectorcardiogram is associated with propagation of the cardiac activation wavefront along (that is, parallel or tangent to) the cardiac surface that is substantially homogeneous through the thickness of the cardiac wall (e.g., with little or no transmural propagation or variation). This sort of homogeneous propagation of the cardiac activation wavefront is not generally considered pathological. This is illustrated, for example, in vectorcardiogram loops 400a, 400b and corresponding time interval plot 402.

Time interval plots 404, 406, on the other hand, have low planarity. Low planarity is associated with pathological tissue. Time interval plot 404, for example, is a highly complex pattern indicative of multiple deflections. This suggests that the underlying electrophysiological data were measured at or near points of variable delay and direction, collision, or lines of isolation.

Time interval plot 406 likewise has low planarity, but, unlike the complexity in time interval plot 404, is substantially linear (that is, one-dimensional). Moreover, time interval plot 406 is substantially perpendicular to the cardiac surface. Together, the linear nature of time interval plot 404 and its direction relative to the cardiac surface suggest at least some propagation of the cardiac activation wavefront through the myocardium (referred to herein as “transmural conduction”), rather than substantially along the cardiac surface, where the underlying electrophysiology data were collected. This pathology is often associated with focal sources and connections.

The planarity of the vectorcardiogram shape can be analyzed using singular value decomposition (SVD). Those of ordinary skill in the art will be familiar with SVD techniques. By way of illustration, however, one suitable approach is to compare the least singular value to a function of the two largest singular values (for example, the product of the two larger singular values), scaled by the geometric mean of singular values from the point cloud of the vectorcardiogram. The planarity of the vectorcardiogram can, in turn, be measured as a ratio of the geometric mean of the two greatest singular values to the least singular value.

Insofar as the planarity of the vectorcardiogram can be indicative of whether the underlying cardiac tissue is healthy or pathological, it is contemplated that the planarity of the vectorcardiogram shape can be tested against one or more planarity (or, in certain contexts, non-planarity) criteria as part of assessing the likelihood that the underlying cardiac tissue is pathological. For instance, where the magnitude in the least deflection direction or singular value is between about 10% and about 20% of the others (or, alternatively, the geometric mean of the others), one can reasonably conclude that the vectorcardiogram is highly planar (that is, substantially two-dimensional). As mentioned above, a vectorcardiogram that is substantially constrained to a single plane (e.g., that has high planarity) reflects a high degree of homogeneous cardiac activation wavefront propagation, as observed in healthy tissue with propagation along the cardiac surface, allowing for a conclusion that the underlying cardiac tissue is not pathological.

On the other hand, where the most prominent deflection or singular value is about three or more times greater than the others, one can reasonably conclude that the vectorcardiogram is substantially one-dimensional. In this instance, and as discussed in further detail below, the direction(s) of the vectorcardiogram relative to the cardiac surface can be instructive to consideration of whether the underlying cardiac tissue is pathological or not.

Finally, where all deflections or singular values are within about 20% to about 30% of their collective geometric mean, one can reasonably conclude that the vectorcardiogram is three-dimensional, rather than one-or two-dimensional.

With respect to analyzing the direction of the vectorcardiogram relative to the cardiac tissue, a threshold angle between the vectorcardiogram and the cardiac surface can be used to determine whether and/or to what extent the underlying tissue is pathological. For instance, if the angle between the vectorcardiogram and the cardiac surface is less than about 20 degrees, the underlying tissue may be classified as non-pathological. If the angle between the vectorcardiogram and the cardiac surface is between about 20 degrees and about 30 degrees, the underlying tissue may be classified as weakly pathological. If the angle between the vectorcardiogram and the cardiac surface is greater than about 30 degrees, the underlying tissue may be classified as strongly pathological. These various thresholds are examples of what are referred to herein as “directional criteria.”

In another aspect of block 306, system 8 can analyze the electrophysiological activity described by the vectorcardiogram. Various publications, including those incorporated above, describe in detail the computation of E-field loops (analogous to vectorcardiograms) and omnipolar electrograms for two-and three-dimensional electrode cliques. Thus, a detailed discussion of how omnipolar electrograms are computed for a given electrode clique is not necessary to an understanding of the instant disclosure. Instead, it will suffice to mention that, for a given electrode clique, the corresponding vectorcardiogram describes the set of omnipolar electrograms for such clique across all possible omnipole (virtual bipole) orientations, and that this set of omnipolar electrograms can, in turn, be analyzed with respect to numerous electrophysiological characteristics that may be of interest to a practitioner in particular contexts.

For example, the omnipolar electrograms described by the clique vectorcardiogram may be analyzed, under an assumption that the cardiac activation wavefront behaves as a traveling wave, to determine a three-dimensional activation direction for the underlying tissue. This can be achieved, for example, through morphological matching between signals (e.g., by identifying the omnipolar electrogram that has the best morphological match to a time derivative of a unipolar electrogram for the electrode clique or identifying the omnipolar electrogram integral that has the best morphological match to a unipolar electrogram for the electrode clique). Suitable techniques for defining unipolar electrograms for cliques of electrodes and for identifying morphological matches between electrograms for cliques of electrodes (and thus identifying the traveling wave activation direction) are described in U.S. Pat. No. 11,751,794, which is hereby incorporated by reference as though fully set forth herein.

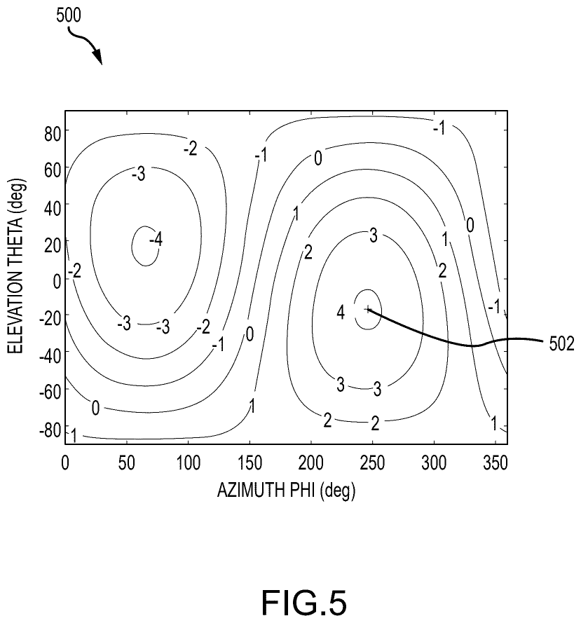

To illustrate an approach to identifying the traveling wave activation direction, FIG. 5 depicts a representative contour plot 500 of the inner product between the omnipolar and unipolar electrograms for a given electrode clique. The three-dimensional omnipolar orientation in FIG. 5 is expressed in terms of the angle components Θ (elevation) and Φ (azimuth) of a spherical coordinate system. Contour plot 500 has maximum 502 at about −20degrees elevation and about 240 degrees azimuth; within the context of FIG. 5, this is the traveling wave activation direction.

Those of ordinary skill in the art will, of course, be familiar with other techniques for computing morphological matches between electrogram signals (e.g., attribute matching, template matching, frequency content matching, and the like). It is contemplated that any such techniques can be applied to identify the traveling wave activation direction for the underlying tissue.

As another example, the omnipolar electrograms described by the clique vectorcardiogram may be analyzed to identify the orientation(s) where the omnipolar electrogram exhibits maximum fractionation or maximum complexity. One suitable measure of complexity is the number of local extremes over an analysis interval, weighted by the logarithmic amplitude of each extreme from a measure of central tendency (e.g., mean, median) for the interval, though other measures of complexity are regarded as within the scope of the instant disclosure.

As yet another example, the omnipolar electrograms described by the clique vectorcardiogram may be analyzed to identify the orientation(s) of maximum peak frequency.

As a still further example, the omnipolar electrograms described by the clique vectorcardiogram may be analyzed to identify orientation(s) that maximize the recognition of two (or more) distinct deflections in the omnipolar electrogram signal(s). This is referred to herein as a “split potential.” If the split potentials have sufficiently different orientations, it may indicate that the underlying tissue is pathological in that it is capable of sustaining multiple independent conduction pathways simultaneously. In some embodiments of the disclosure, acute angular separations of between about 30 degrees and about 90 degrees can be interpreted as indicative of split potentials, whereas angular separations of between about 0 degrees and about 30 degrees can be interpreted otherwise.

The ordinarily-skilled artisan will recognize that the foregoing electrophysiological characteristics that may be understood from omnipolar electrograms are illustrative rather than exhaustive. In certain applications of the teachings herein, it may be desirable to analyze other electrophysiological characteristics for insights into whether and/or to what extent the underlying tissue is or may be pathological.

Another aspect of the instant disclosure relates to computing cardiac activation vectors. To this end, in block 308, system 8 can identify at least a first omnipolar electrogram and a second omnipolar electrogram for the electrode clique. As noted above, those of ordinary skill in the art will appreciate how to compute omnipolar electrograms for the electrode clique, and it will suffice for an understanding of the instant disclosure to describe the how the first omnipolar electrogram and second omnipolar electrogram are selected from amongst possible candidates (e.g., as between all possible omnipolar electrograms for a given electrode clique).

In certain aspects of the disclosure, the first omnipolar electrogram is a maximum peak-to-peak voltage omnipolar electrogram. That is, of all possible omnipolar electrograms for a given electrode clique, the one having the highest peak-to-peak voltage is defined as the first omnipolar electrogram for the clique.

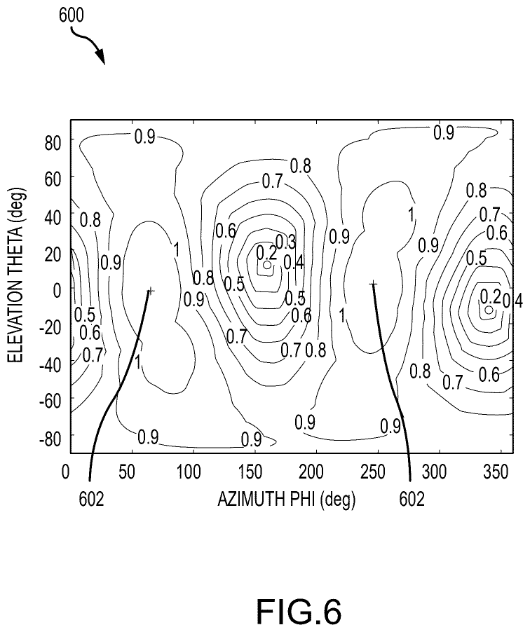

FIG. 6 illustrates identifying the maximum peak-to-peak voltage omnipolar electrogram via a representative contour plot 600 of omnipolar electrogram peak-to-peak voltages. In FIG. 6, as in FIG. 5, the three-dimensional omnipole orientation is expressed in terms of the angle components Θ (elevation) and Φ (azimuth) of a spherical coordinate system. Contour plot 600 shows two maximum peak-to-peak voltages 602, one at about 0 degrees elevation and about 60 degrees azimuth and a second at about 0 degrees elevation and about 240 degrees azimuth (the ordinarily-skilled artisan will appreciate that two maxima occur because the peak-to-peak bipole orientations are antipodal or diametrically opposed with 180 degrees of ambiguity).

In other aspects of the disclosure, the first omnipolar electrogram is selected according to an electrophysiological characteristic other than peak-to-peak voltage. For instance, it is contemplated that any of the following could instead be defined as the first omnipolar electrogram: the omnipolar electrogram having the lowest peak frequency in the wavelet domain; the omnipolar electrogram having the greatest peak frequency in the wavelet domain; the omnipolar electrogram having the lowest depolarization complexity index; or the omnipolar electrogram having the greatest depolarization complexity index. It should be understood, however, that the foregoing characteristics are merely exemplary, and that other characteristics could likewise be used to good advantage in connection with the present teachings.

Note, however, that the foregoing electrophysiological characteristics are merely exemplary of those that could be used to define the first omnipolar electrogram and are not intended to be an exhaustive listing of such characteristics. Indeed, those of ordinary skill in the art will recognize the usefulness of additional and/or different electrophysiological characteristics when defining the first omnipolar electrogram for the electrode clique.

Turning now to the second omnipolar electrogram for the electrode clique, it is contemplated to identify the omnipolar electrogram that has the best morphological match to a time derivative of a unipolar electrogram for the electrode clique. Corresponding techniques are described above in connection with the traveling wave activation direction analysis of the clique vectorcardiogram.

Once the first and second omnipolar electrograms for the clique are identified in block 308, an activation direction for the electrode clique is defined in block 310. According to embodiments of the disclosure, at least one of the orientation of the first omnipolar electrogram and the orientation of the second omnipolar electrogram are used to define the activation direction for the clique.

For healthy tissue, these two orientations will typically be relatively close to each other, with between about 0 degrees and about 10 degrees offset. On the other hand, if the offset is larger, such as greater than about 15 degrees, it is an indication that the underlying tissue is pathological (that is, arrhythmia-sustaining).

It is also the case that other discrepancies between the first omnipolar electrogram and the second omnipolar electrogram, such as considerable differences in amplitude or activation time, may be indicative of underlying pathology, insofar as marked differences in direction between the first omnipolar electrogram and the second omnipolar electrogram may reflect that the underlying tissue is capable of sustaining multiple independent conduction pathways.

In any event, the activation direction for the electrode clique can be defined as the orientation of the first omnipolar electrogram, as the orientation of the second omnipolar electrogram, or as a hybrid of the orientations of the first and second electrograms (e.g., the average of the two orientations). As with FIGS. 5 and 6, the activation direction for the electrode clique can be expressed in terms of the angle components Θ (elevation) and Φ (azimuth) of a spherical coordinate system.

In block 312, system 8 computes a conduction velocity magnitude for the electrode clique. In certain aspects of the disclosure, the conduction velocity magnitude is computed as a ratio between the amplitude of an omnipolar electrogram for the clique (e.g., the first omnipolar electrogram for the clique, the second omnipolar electrogram for the clique, or a hybrid of the first and second omnipolar electrograms for the clique) and a rate of change in a unipolar electrogram for the clique. This is described, for example, in Deno, referenced above.

Alternatively, the conduction velocity magnitude can be computed as a ratio between an integral of an omnipolar electrogram for the clique (e.g., the first omnipolar electrogram for the clique, the second omnipolar electrogram for the clique, or a hybrid of the first and second omnipolar electrograms for the clique) and the amplitude of a unipolar electrogram for the clique. This is described, for example, in U.S. Pat. No. 11,751,794, referenced above.

Collectively, the activation direction computed in block 310 and the conduction velocity magnitude computed in block 312 define a cardiac activation vector at the cardiac location at which the electrophysiological data were measured. By repeating blocks 302 through 312 at several different cardiac locations, a cardiac activation map (e.g., a collection of cardiac activation vectors at various locations over the cardiac surface) can be generated and, if desired, output to display 23 using known techniques (e.g., as a collection of arrow icons or other glyphs superimposed upon a three-dimensional geometric model of the heart).

In block 314, system 8 can classify the underlying tissue by pathology (e.g., as pathological or not pathological) based on the vectorcardiogram analyses and/or omnipolar electrogram definitions from the preceding blocks. For instance, if the vectorcardiogram shape satisfies a non-planarity criterion, a directional criterion, and/or a complexity criterion (examples of which are described above), then the cardiac location can be classified as pathological.

As another example, if one or more characteristics of the first omnipolar electrogram and the second omnipolar electrogram differ by significant amounts (an example is given above for discrepancies in activation direction as between the two), then the cardiac location can be classified as pathological.

In block 316, system 8 outputs a graphical representation of the cardiac activation vector at the cardiac location. As briefly mentioned above, this can be in the form of an arrow icon or other glyph that corresponds to the direction and magnitude of the cardiac activation vector and that is superimposed on a three-dimensional model of the cardiac geometry.

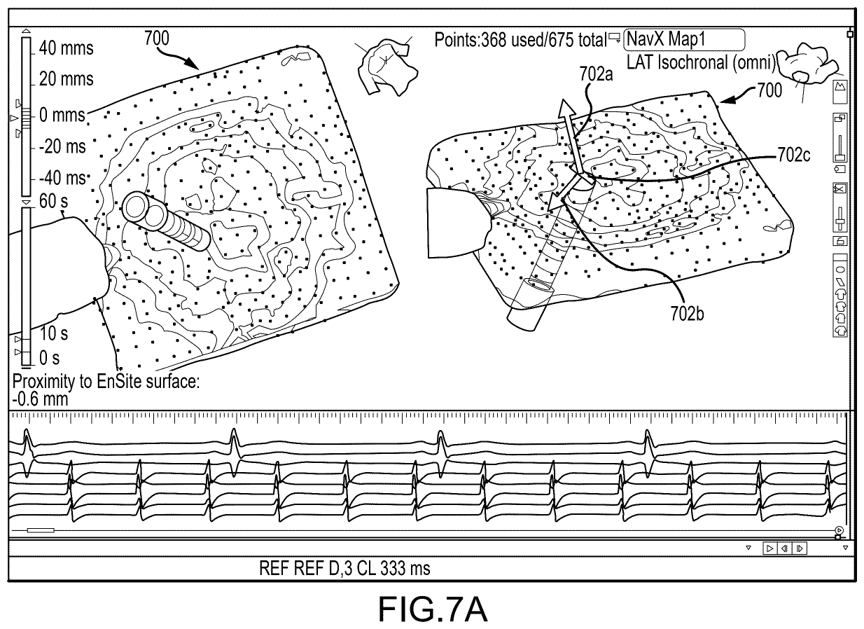

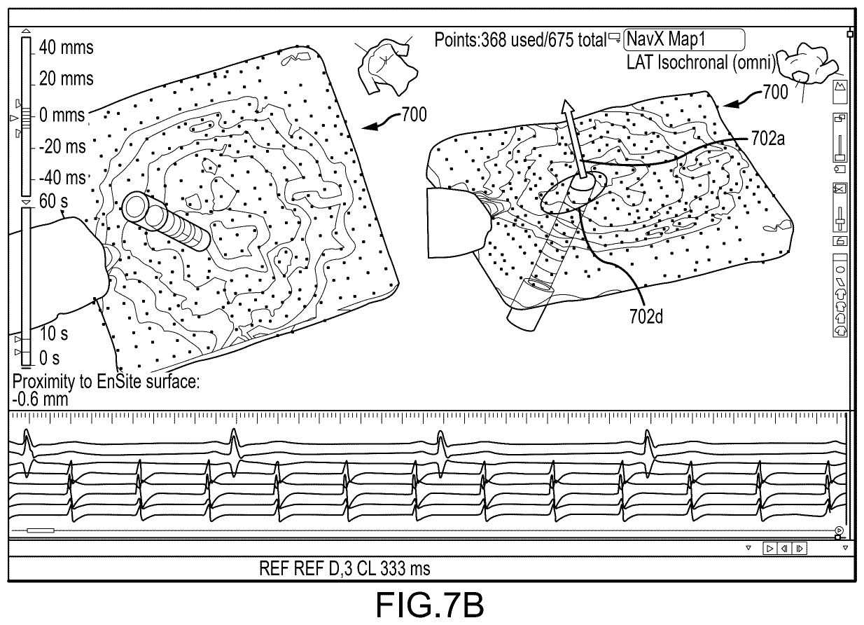

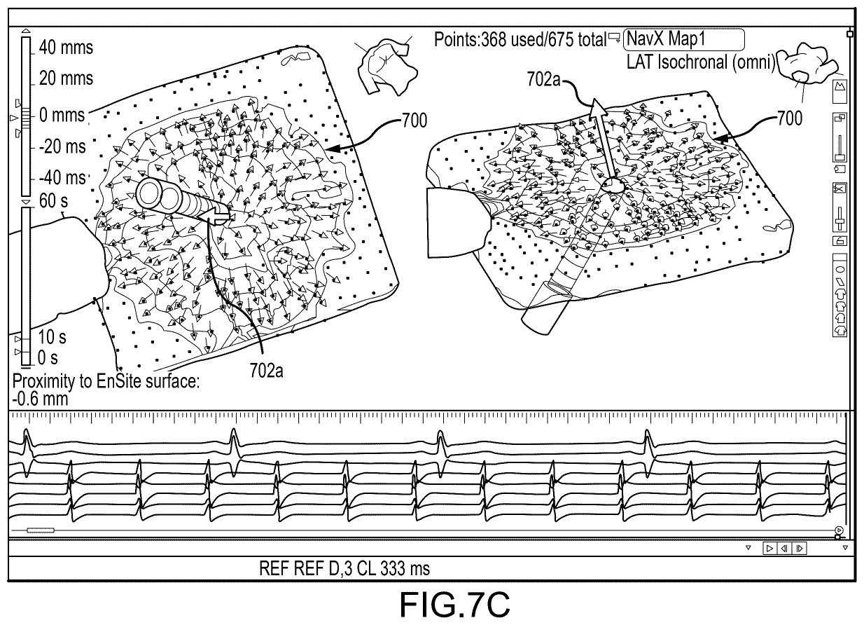

Extant representations of cardiac activation vectors, such as arrow icons 700 in FIG. 7C, are constrained to the cardiac surface, because the analysis is constrained to two dimensions and ignores transmural conduction. As described above, however, three-dimensional omnipolar electrograms can also detect transmural conduction (that is, the component of a cardiac activation vector that is normal to the plane of the cardiac surface). Revealing transmural conduction as described enables improved characterization of cardiac activation with commensurate advantages for subsequent treatment. Moreover, some characteristics of cardiac activation that are otherwise not revealed when analysis is constrained to two dimensions may be revealed by taking a three-dimensional approach.

A representative graphical output of a cardiac activation vector in three dimensions at the location of catheter 13 is shown as arrow icons 702a and 702b in FIG. 7A. In particular, arrow icon 702a shows the component of the cardiac activation vector that is normal to the cardiac surface, while arrow icon 702b shows the component of the cardiac activation vector that is tangent to the cardiac surface. The colors and/or relative sizes (e.g., length, line weight, thickness, and/or the like) of arrow icons 702a, 702b can be adjusted to reflect the relative magnitudes of these respective components. An additional line segment 702c can be used to designate a component perpendicular to the plane the activation direction and/or vectorcardiogram loop occupy.

FIG. 7B depicts an alternative graphical output of a cardiac activation vector in three dimensions. In FIG. 7B, the combination of arrow icon 702b and line segment 702c are reflected in an ellipse 702d. The major axis of ellipse 702d corresponds to arrow icon 702b, while the minor axis of ellipse 702d corresponds to line segment 702c.

An advantageous aspect of the instant disclosure is that the graphical representation of the cardiac activation vector may appear in three-dimensions, rather than being constrained to the plane of the cardiac surface. Where this graphical representation has a substantial component normal to the plane of the cardiac surface, it can indicate that the underlying tissue may be pathological and may therefore be a desirable location for delivery of ablation therapy.

In aspects of the disclosure these three-dimensional cardiac activation vector representations appear only at the present location of catheter 13 in a real-time fashion (e.g., continuously updated for the current beat). Optionally, they may persist for a relatively short period of time following the current beat. For example, they may fade out over a fixed time period (e.g., about two seconds) or over a time period that correlates to local conduction velocity (e.g., fading slowly where local conduction velocity is low and more quickly where local conduction velocity is higher). In general, however, to minimize visual clutter, three-dimensional cardiac activation vector representations for prior locations of catheter 13 and/or for prior beats do not persist for extended periods of time.

Another contemplated approach to minimizing visual clutter is to display a three-dimensional cardiac activation vector representation only if analysis determines that the underlying tissue may be pathological (in other words, a three-dimensional cardiac activation vector representation is not displayed when it would be substantially along the cardiac surface with little or no transmural conduction component). Such locations can be identified by checking whether the cardiac activation vector satisfies a transmural conduction criterion, such as when the angle between the cardiac activation vector and the cardiac surface exceeds a preset threshold. This threshold may be the same as, similar to, or different from the directional criterion threshold angle discussed above. If, on the other hand, the cardiac activation vector does not satisfy the transmural conduction criterion, the three-dimensional cardiac activation vector can be projected onto the cardiac surface in two-dimensions and displayed accordingly (e.g., using techniques and conventions that will be familiar to those of ordinary skill in the art).

An exemplary such output is shown in FIG. 7C. As shown in FIG. 7C, conduction velocity vectors along the cardiac surface are illustrated using arrow icons 700 (as will be familiar to those of ordinary skill in the art). As those of ordinary skill in the art will appreciate, arrow icons 700 are typically persistent. At the location of arrow icon 702a, however, the conduction velocity has a sufficiently large component normal to the surface to merit illustration. In some aspects of the disclosure, it may be desirable for three-dimensional cardiac activation vector representations that are indicative of pathological underlying tissue to persist, such that the representation of cardiac activation continuously highlights potential ablation targets. In some embodiments of the disclosure, therefore, the three-dimensional cardiac activation vector representation (e.g., arrow 702a in FIG. 7C) can also persist when it satisfies a transmural conduction criterion.

Further, the appearance of the three-dimensional cardiac activation vector representation can provide additional information about the magnitude and/or direction of the cardiac activation vector. For instance, the length or thickness of the representation can increase with increasing conduction velocity. As another example, the color of the representation can vary according to the angle between the cardiac activation vector and the cardiac surface, shifting from green, to yellow, to red as that angle increases, to reflect greater degrees of pathology.

Although several embodiments have been described above with a certain degree of particularity, those skilled in the art could make numerous alterations to the disclosed embodiments without departing from the spirit or scope of this invention.

For example, the teachings herein can be applied in real time (e.g., during an electrophysiology study) or during post-processing (e.g., to electrophysiology signals collected during an electrophysiology study performed at an earlier time).

As another example, the graphical output can include not only vector representations (e.g., direction and/or magnitude), but also scalar and/or time series signal representations.

As yet another example, in addition to computing conduction velocity magnitudes (e.g., in block 312) in units of distance traveled per unit of time (e.g., mm/msec), it can be informative to compute magnitudes in units of time required to travel a particular distance (e.g., msec/mm).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

### Numbered Clauses

1. A method of mapping cardiac electrophysiological activity comprising:
   receiving, at an electroanatomical mapping system, electrophysiological data from at least four non-coplanar electrodes carried by a multi-electrode catheter positioned at a cardiac location, the at least four non-coplanar electrodes defining a three-dimensional electrode clique; and
   the electroanatomical mapping system executing a process comprising:
      deriving a three-dimensional vectorcardiogram for the three-dimensional electrode clique;
      analyzing a shape of the three-dimensional vectorcardiogram;
      identifying a first omnipolar electrogram for the three-dimensional electrode clique, wherein the first omnipolar electrogram is a maximum peak-to-peak voltage omnipolar electrogram;
      identifying a second an omnipolar electrogram for the three-dimensional electrode clique, wherein the second omnipolar electrogram has a best morphological match to a unipolar electrogram for the three-dimensional electrode clique;
      using at least one of an orientation of the first omnipolar electrogram and an orientation of the second omnipolar electrogram to define an activation direction for the three-dimensional electrode clique; and
      computing a conduction velocity magnitude for the three-dimensional electrode clique,
   thereby determining a cardiac activation vector at the cardiac location.
2. The method according to clause 1, wherein the process executed by the electroanatomical mapping system further comprises classifying the cardiac location as pathological when the orientation of the first omnipolar electrogram differs from the orientation of the second omnipolar electrogram by more than a threshold amount.
3. The method according to clause 1 or clause 2, wherein the process executed by the electroanatomical mapping system further comprises classifying the cardiac location as pathological when the shape of the three-dimensional vectorcardiogram satisfies at least one of a non-planarity criterion and a directional criterion.
4. The method according to clause 3, wherein analyzing the shape of the three-dimensional vectorcardiogram comprises analyzing at least one of planarity of the three-dimensional vectorcardiogram and an angle of the three-dimensional vectorcardiogram relative to a cardiac surface at the cardiac location.
5. The method according to clause 4, wherein analyzing the planarity of the three-dimensional vectorcardiogram comprises analyzing the planarity of the three-dimensional vectorcardiogram using singular value decomposition.
6. The method according to any one of clauses 1 to 5, further comprising the electroanatomical mapping system outputting a graphical representation of the cardiac activation vector at the cardiac location.
7. The method according to clause 6, wherein the electroanatomical mapping system outputting the graphical representation of the cardiac activation vector at the cardiac location comprises the electroanatomical mapping system outputting the graphical representation of the cardiac activation vector at the cardiac location when the cardiac activation vector satisfies a transmural conduction criterion.
8. The method according to clause 7, wherein the transmural conduction criterion comprises a threshold for an angle between the activation direction and a cardiac surface at the cardiac location.
9. The method according to any one of clauses 1 to 8, wherein the at least four non-coplanar electrodes carried by the multi-electrode catheter comprise a segmented tip electrode and a ring electrode, wherein the segmented tip electrode includes at least three segments.
10. The method according to any one of clauses 1 to 8, wherein the at least four non-coplanar electrodes carried by the multi-electrode catheter comprise a tip electrode and a segmented ring electrode, wherein the segmented ring electrode includes at least three segments.
11. The method according to any one of clauses 1 to 8, wherein the at least four non-coplanar electrodes carried by the multi-electrode catheter comprise a segmented tip electrode and a segmented ring electrode.
12. An electroanatomical mapping system for generating a cardiac activation map, comprising:
   a cardiac activation module configured to:
      receive electrophysiological data from at least four non-coplanar electrodes carried by a multi-electrode catheter positioned at a cardiac location, the at least four non-coplanar electrodes defining a three-dimensional electrode clique;
      derive a three-dimensional vectorcardiogram for the three-dimensional electrode clique;
      analyze a shape of the three-dimensional vectorcardiogram;
      identify a first omnipolar electrogram for the three-dimensional electrode clique, wherein the first omnipolar electrogram is a maximum peak-to-peak voltage omnipolar electrogram;
      identify a second omnipolar electrogram for the three-dimensional electrode clique, wherein the second omnipolar electrogram has a best morphological match to a unipolar electrogram for the three-dimensional electrode clique;
      define an activation direction for the three-dimensional electrode clique using at least one of an orientation of the first omnipolar electrogram and an orientation of the second omnipolar electrogram;
      compute a conduction velocity magnitude for the three-dimensional electrode clique; and
      associate the activation direction and the conduction velocity magnitude as a cardiac activation vector at the cardiac location.
13. The electroanatomical mapping system according to clause 12, wherein the cardiac activation module is further configured to classify the cardiac location as pathological when the orientation of the first omnipolar electrogram differs from the orientation of the second omnipolar electrogram by more than a threshold amount.
14. The electroanatomical mapping system according to clause 12 or clause 13, wherein the cardiac activation module is further configured to classify the cardiac location as pathological when the shape of the three-dimensional vectorcardiogram satisfies at least one of a non-planarity criterion and a directional criterion.
15. The electroanatomical mapping system according to clause 14, wherein the cardiac activation module is configured to analyze the shape of the three-dimensional vectorcardiogram by analyzing at least one of a planarity of the three-dimensional vectorcardiogram and an angle of the three-dimensional vectorcardiogram relative to a cardiac surface at the cardiac location.
16. The electroanatomical mapping system according to clause 15, wherein the cardiac activation module is configured to analyze the planarity of the three-dimensional vectorcardiogram using singular value decomposition.
17. The electroanatomical mapping system according to any one of clauses 12 to 16, wherein the cardiac activation module is further configured to output a graphical representation of the cardiac activation vector at the cardiac location.
18. The electroanatomical mapping system according to clause 17, wherein the graphical representation of the cardiac activation vector at the cardiac location comprises an arrow icon superimposed upon a three-dimensional model of a cardiac geometry.
19. The electroanatomical mapping system according to clause 18, wherein the graphical representation of the cardiac activation vector at the cardiac location is only output when the cardiac activation vector satisfies a transmural conduction criterion.
20. The electroanatomical mapping system according to clause 19, wherein the transmural conduction criterion comprises a threshold for an angle between the activation direction and a cardiac surface at the cardiac location.

What is claimed is:

1. A method of mapping cardiac electrophysiological activity comprising:
receiving, at an electroanatomical mapping system, electrophysiological data from at least four non-coplanar electrodes positioned at a cardiac location, the at least four non-coplanar electrodes defining a three-dimensional electrode clique; and
the electroanatomical mapping system executing a process comprising:
deriving a three-dimensional vectorcardiogram for the three-dimensional electrode clique;
analyzing a shape of the three-dimensional vectorcardiogram;
identifying a first omnipolar electrogram for the three-dimensional electrode clique, wherein the first omnipolar electrogram is a maximum peak-to-peak voltage omnipolar electrogram;
identifying a second an omnipolar electrogram for the three-dimensional electrode clique, wherein the second omnipolar electrogram has a best morphological match to a unipolar electrogram for the three-dimensional electrode clique;
using at least one of an orientation of the first omnipolar electrogram and an orientation of the second omnipolar electrogram to define an activation direction for the three-dimensional electrode clique; and
computing a conduction velocity magnitude for the three-dimensional electrode clique, thereby determining a cardiac activation vector at the cardiac location.

2. The method according to claim 1, wherein the process executed by the electroanatomical mapping system further comprises classifying the cardiac location as pathological when the orientation of the first omnipolar electrogram differs from the orientation of the second omnipolar electrogram by more than a threshold amount.

3. The method according to claim 1, wherein the process executed by the electroanatomical mapping system further comprises classifying the cardiac location as pathological when the shape of the three-dimensional vectorcardiogram satisfies at least one of a non-planarity criterion and a directional criterion.

4. The method according to claim 3, wherein analyzing the shape of the three-dimensional vectorcardiogram comprises analyzing at least one of planarity of the three-dimensional vectorcardiogram and an angle of the three-dimensional vectorcardiogram relative to a cardiac surface at the cardiac location.

5. The method according to claim 4, wherein analyzing the planarity of the three-dimensional vectorcardiogram comprises analyzing the planarity of the three-dimensional vectorcardiogram using singular value decomposition.

6. The method according to claim 1, further comprising the electroanatomical mapping system outputting a graphical representation of the cardiac activation vector at the cardiac location.

7. The method according to claim 6, wherein the electroanatomical mapping system outputting the graphical representation of the cardiac activation vector at the cardiac location comprises the electroanatomical mapping system outputting the graphical representation of the cardiac activation vector at the cardiac location when the cardiac activation vector satisfies a transmural conduction criterion.

8. The method according to claim 7, wherein the transmural conduction criterion comprises a threshold for an angle between the activation direction and a cardiac surface at the cardiac location.

9. The method according to claim 1, wherein the at least four non-coplanar electrodes comprise a segmented tip electrode and a ring electrode carried by a multi-electrode catheter, wherein the segmented tip electrode includes at least three segments.

10. The method according to claim 1, wherein the at least four non-coplanar electrodes comprise a tip electrode and a segmented ring electrode carried by a multi-electrode catheter, wherein the segmented ring electrode includes at least three segments.

11. The method according to claim 1, wherein the at least four non-coplanar electrodes comprise a segmented tip electrode and a segmented ring electrode carried by a multi-electrode catheter.

12. An electroanatomical mapping system for generating a cardiac activation map, comprising:
a cardiac activation module configured to:
receive electrophysiological data from at least four non-coplanar electrodes positioned at a cardiac location, the at least four non-coplanar electrodes defining a three-dimensional electrode clique;
derive a three-dimensional vectorcardiogram for the three-dimensional electrode clique;
analyze a shape of the three-dimensional vectorcardiogram;
identify a first omnipolar electrogram for the three-dimensional electrode clique, wherein the first omnipolar electrogram is a maximum peak-to-peak voltage omnipolar electrogram;
identify a second omnipolar electrogram for the three-dimensional electrode clique, wherein the second omnipolar electrogram has a best morphological match to a unipolar electrogram for the three-dimensional electrode clique;
define an activation direction for the three-dimensional electrode clique using at least one of an orientation of the first omnipolar electrogram and an orientation of the second omnipolar electrogram;
compute a conduction velocity magnitude for the three-dimensional electrode clique; and
associate the activation direction and the conduction velocity magnitude as a cardiac activation vector at the cardiac location.

13. The electroanatomical mapping system according to claim 12, wherein the cardiac activation module is further configured to classify the cardiac location as pathological when the orientation of the first omnipolar electrogram differs from the orientation of the second omnipolar electrogram by more than a threshold amount.

14. The electroanatomical mapping system according to claim 12, wherein the cardiac activation module is further configured to classify the cardiac location as pathological when the shape of the three-dimensional vectorcardiogram satisfies at least one of a non-planarity criterion and a directional criterion.

15. The electroanatomical mapping system according to claim 14, wherein the cardiac activation module is configured to analyze the shape of the three-dimensional vectorcardiogram by analyzing at least one of a planarity of the three-dimensional vectorcardiogram and an angle of the three-dimensional vectorcardiogram relative to a cardiac surface at the cardiac location.

16. The electroanatomical mapping system according to claim 15, wherein the cardiac activation module is configured to analyze the planarity of the three-dimensional vectorcardiogram using singular value decomposition.

17. The electroanatomical mapping system according to claim 12, wherein the cardiac activation module is further configured to output a graphical representation of the cardiac activation vector at the cardiac location.

18. The electroanatomical mapping system according to claim 17, wherein the graphical representation of the cardiac activation vector at the cardiac location comprises an arrow icon superimposed upon a three-dimensional model of a cardiac geometry.

19. The electroanatomical mapping system according to claim 17, wherein the graphical representation of the cardiac activation vector at the cardiac location is only output when the cardiac activation vector satisfies a transmural conduction criterion.

20. The electroanatomical mapping system according to claim 19. wherein the transmural conduction criterion comprises a threshold for an angle between the activation direction and a cardiac surface at the cardiac location.
